(19) United States
(12) Patent Application Publication
(10) Pub. No.: US 2023/0001078 A1
(43) Pub. Date: Jan. 5, 2023

(54) SEAL BETWEEN HOUSING SHELLS

(71) Applicant: B. Braun Melsungen AG, Melsungen (DE)

(72) Inventors: Jan Schwarz, Melsungen (DE); Stephan Hoevel, Kassel (DE); Matthias Schwalm, Schwalmstadt (DE); Dirk Aljets, Wolfhagen (DE); Joachim Schuetz, Fulda (DE)

(21) Appl. No.: 17/849,654

(22) Filed: Jun. 26, 2022

(30) Foreign Application Priority Data
Jul. 1, 2021 (DE) 20 2021 103 525.0

(51) Int. Cl. A61M 5/142

(52) U.S. Cl. CPC A61M 5/14228; A61M 2205/0216

(57) ABSTRACT

A medical pump, preferably a peristaltic pump or a syringe pump, for conveying a medical fluid. The pump includes a housing with an upper housing shell, a lower housing shell and a front lid, and a seal between the upper housing shell and the lower housing shell. The front lid is pivotably arranged on the lower housing shell.

### CROSS-REFERENCE TO RELATED APPLICATION

This application claims priority under 35 U.S.C. § 119 to German Application No. 20 2021 103 525.0, filed Jul. 1, 2021, the contents of which is incorporated by reference herein in its entirety.

### FIELD

The disclosure relates to a medical pump made of a plurality of housing shells.

### BACKGROUND

Peristaltic pumps and syringe pumps are used to automatically administer a specific dose of medication to patients. It is known to separate a pump housing into a lower housing shell and an upper housing shell and to join these two shells together during assembly. This facilitates the assembly of the components inside the housing and also service, maintenance and repairs of the pumps. Such a peristaltic pump is disclosed, for example, in CN 2 11 485 985 U.

Medical pumps are exposed to external influences during operation. Particularly noteworthy are fluids that flow out of a leaking or burst hose, for example, or other fluids to which the pump is exposed in normal hospital use. Furthermore, the pumps are exposed to contaminants, such as dust. Both fluids and dust or other contaminants can enter the interior of the housing and damage the pump's electronics, thus affecting the pump's operation. This is especially critical since the pumps deliver vital medications to patients in some cases and should therefore be particularly fail-safe. Known medical pumps are susceptible to damage from fluids and contaminants that can enter the pump housing.

### SUMMARY

Thus, the object of the disclosure is to provide a medical pump that is robust against contamination and fluids. In particular, the pump should ensure at least the IP44 protection type/standard of protection.

The disclosure relates in summary to a medical pump, preferably a peristaltic pump or a syringe pump, for conveying a medical fluid, having a housing which has an upper housing shell, a lower housing shell and a front lid pivotably arranged on the housing, preferably on the lower housing shell. A seal is provided between the two housing shells which seals the housing against contamination and fluids.

The seal is mounted between a plastic shell, which is a housing part of the pump housing, and a further plastic shell, which is another housing part of the pump housing. The seal seals the pump housing against external influences. The seal extends the service life of the pump. Fluids, contaminants and external influences can generally damage the pump, in particular the electronics of the pump. However, since the seal reliably prevents these interfering influences from entering the inside of the pump, the pump has a longer service life and is generally more robust against external influences.

The connection of the housing parts, which are assembled to a housing, and the seal, which is prepared between the housing parts, is suitable for the housing to meet at least the IP44 protection class.

According to a further preferred feature of the disclosure, the seal is a sealing bead that is preferably made of polyurethane. Polyurethane as a sealant has several advantages:
Polyurethane has been used as a sealant for some time. Therefore, the processing and material properties of polyurethane are well known. Polyurethane is foamed, is therefore very flexible in shaping and is elastic.

According to a further preferred feature of the disclosure, the seal is elastic during assembly and disassembly of the housing shells. After disassembly of the two housing shells, for example during repair or maintenance, the seal is still elastic. This allows the two housing shells to be reassembled after repair, and the seal seals the two assembled housing shells without having to change the seal after repair.

According to a further preferred feature of the disclosure, the sealing bead is foamed. The seal is formed by foaming the sealing bead. The foamed seal is distinguished by very good sealing properties. Furthermore, the material of the seal is very light. A wide variety of seal geometries can be achieved by foaming.

According to a further preferred feature of the disclosure, the seal is a tempered seal. The tempering process hardens the sealing foam and the sealing bead is formed. Tempering makes the seal resistant to mechanical damage.

According to a further preferred feature of the disclosure, the seal extends around the entire rim of the housing shell. Since the entire housing is to be sealed, the seal also encompasses the entire circumference of the housing shell. The sealing bead thus extends once completely around the edge of the housing shell. This ensures a gap-free seal and prevents fluid/contamination from entering the housing.

According to another preferred feature of the disclosure, the seal fulfills at least the IP44 protection type. IP44 is a standard that defines the protection type of devices against fluids and dust.

According to a further preferred feature of the disclosure, there is a distance between the housing shells which has a size such that the seal is not impermissibly pressed by the housing shells and a cell structure of the seal is not damaged. Impermissible pressing of the seal is pressing that damages the seal and in particular the cell structure of the seal.

The disclosure preferably provides a method of manufacturing a peristaltic pump having one of the preceding features comprising the following steps:
applying the seal to exactly one first housing shell of the lower housing shell and the upper housing shell,
tempering the seal, and
subsequently placing the other housing shell on the first housing shell.

The seal is only applied to one housing shell. The seal is then tempered. Only after the seal has been tempered is the other housing shell placed on the first housing shell. This allows the two housing shells to be separated for repair without damaging the seal. After repair, the two shells can simply be reassembled and the seal still seals the housing. This saves repair costs and labor time.

The disclosure further provides a method of manufacturing a syringe pump according to one of the preceding features, comprising the following steps:
applying the seal to exactly a first housing shell of the lower housing shell and the upper housing shell,
tempering the seal, and
subsequently placing the other housing shell on the first housing shell
assembling a housing side part to the composite construction of the lower housing shell and the upper housing shell.

As with the peristaltic pump, the seal is only applied to the first housing shell and then tempered. The other housing shell is then placed on top. The housing side part is mounted on the housing. This results in a completely sealed housing for the syringe pump.

### BRIEF DESCRIPTION OF THE DRAWINGS

FIG. 1 shows an isometric view of a peristaltic pump according to a first embodiment;

FIG. 2 shows a top view of an upper housing shell of the peristaltic pump;

FIG. 4 shows an isometric view of a syringe pump according to a second embodiment;

FIG. 5 shows an isometric view of an upper housing shell of the syringe pump; and

FIG. 6 shows a housing side part of the syringe pump.

### DETAILED DESCRIPTION

### First Embodiment

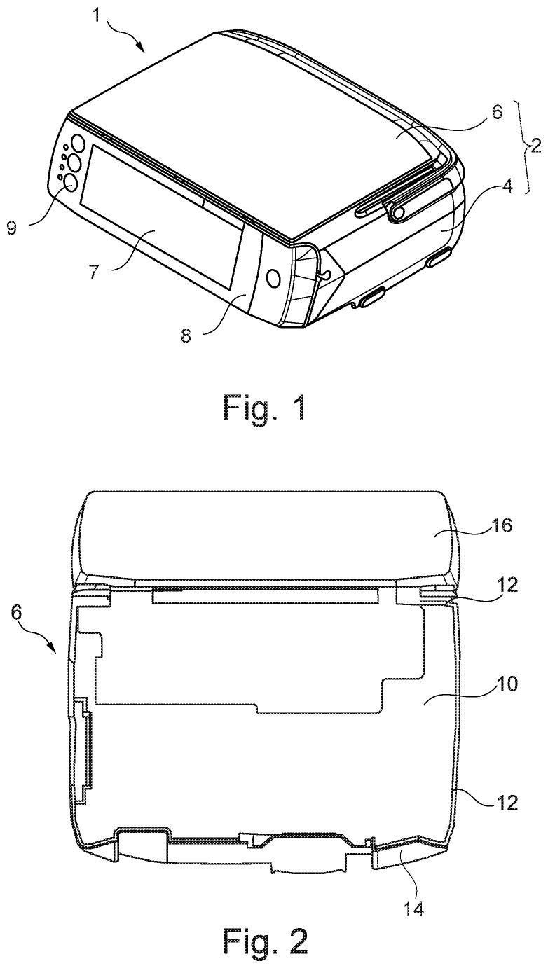

FIG. 1 shows a medical pump 1, in particular a peristaltic pump. However, the medical pump 1 may also be a syringe pump 3 or a dialysis pump. The pump 1 has a housing 2 comprising two housing shells, namely a lower housing shell 4 and an upper housing shell 6. The pump 1 has a front lid 8 that is attached to the lower housing shell 4 and can be folded down. The front lid has a display 7 and buttons 9. Medical pumps such as peristaltic pumps or syringe pumps are generally known, for example from CN 2 11 485 985 U. Therefore, a general description of the mode of operation of the pump is omitted.

FIG. 2 shows a top view of the upper housing shell 6 of the peristaltic pump 1. The upper housing shell 6 is essentially a shell that is open at the top and has an approximately rectangular outline. The upper housing shell 6 has a flat lid 10 from which side walls 14 protrude. The upper housing shell 6 is slightly convex at a rear side of the upper housing shell 6. The rear side is the side on which a connector and a power supply (not shown) of the pump 1 are attached. At a front side opposite to the rear side, the upper housing shell 6 has a projection 16. The projection 16 protects the front lid 8. Inside the housing 2, components of the medical pump 1 are arranged. These are, in part, electronic components such as the power supply, a battery, an electric motor, and electrical wiring and the like (none of which are shown). These components are susceptible to fluids and contaminants. Furthermore, peristaltic pumps 1, for example, include a peristaltic system (not shown) for moving fluid in a tube. The peristaltic system and its drive are sensitive mechanical components and therefore susceptible to contaminations and particles that jam or block moving parts.

A seal 12 runs around the rim of the upper housing shell 6. I.e. the seal 12 runs around the upper rim of the side walls 14. The seal 12 runs around the entire rim. The seal 12 is a sealing bead made of an elastic plastic, preferably polyurethane. The seal 12 is applied by foaming to a housing shell, preferably the upper housing shell 6. After application of the sealing bead, the housing shell is tempered with the sealing bead. The tempering step takes place before the two housing shells are assembled. This ensures that the two housing shells can be taken apart when repairing the pump 1 without damaging the seal 12. The seal 12 is intended to be still resilient after the housing shells have been separated and is intended to seal against contaminations and fluids after reassembly.

FIG. 3 shows a top view of the lower housing shell 4. The lower housing shell 4 is a flat plate with an approximately rectangular outline from which only side walls protrude on the short sides and a battery compartment in the center of the plate. Like the upper housing shell 6, the lower housing shell 4 is convex at the back of the plate, on the side of the power supply. It is shown that no seal is attached to the lower housing shell 4.

### Second Embodiment

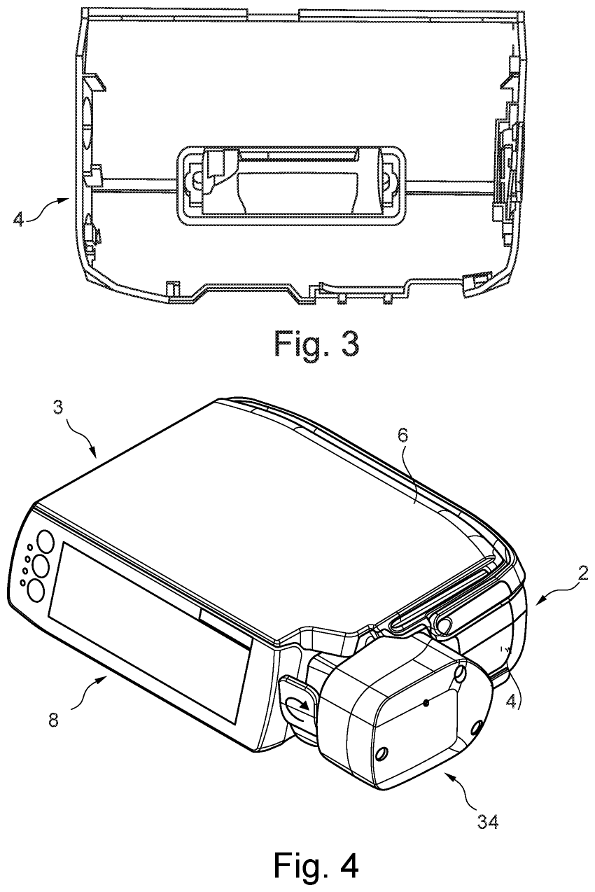

FIG. 4 shows a syringe pump 3. The syringe pump 3 is configured substantially the same as the peristaltic pump 1 of the first embodiment. The syringe pump 3 has a housing 2 with a lower housing shell 4 and an upper housing shell 6. Like the peristaltic pump 1, the syringe pump 3 has a front lid 8. In contrast to the peristaltic pump 1, the syringe pump 3 has a lateral drive head 34. The housing connection of the drive head 34 is shown below in FIG. 6.

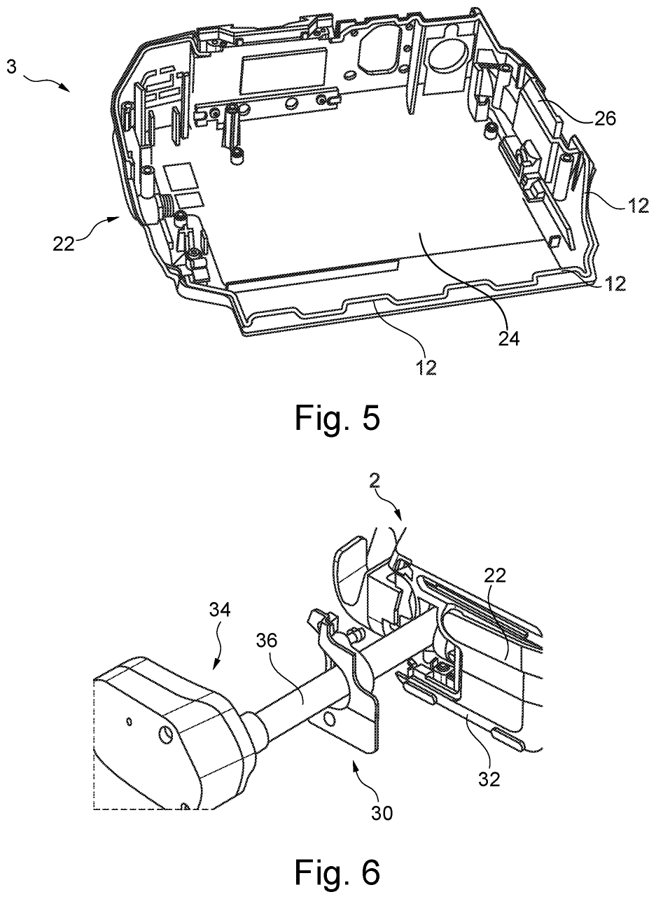

FIG. 5 shows an upper housing shell 22 of the syringe pump 3. The upper housing shell 22 of the syringe pump 3 is similar in design to the upper housing shell 6 of the peristaltic pump 1. The upper housing shell 22 has a flat lid 24 and side walls 26 projecting from the lid 24. Components of the pump, such as a main circuit board (not shown) and the like, are arranged inside the upper housing shell 22. In the syringe pump 3, the seal 12 is also applied to a housing shell, preferably the upper housing shell 22, then tempered, and only then is the other housing shell placed on top. The seal 12 covers the entire rim of the upper housing shell 22.

FIG. 6 shows a housing side part 30 in the syringe pump 3. In a peristaltic pump 1 as in FIGS. 1 to 3, the housing 2 has only the lower housing shell 4 and the upper housing shell 6. In the syringe pump 3, the housing 2 has the lower housing shell 32, the upper housing shell 22, and a housing side part 30. The housing side part 30 is arranged on one side of the housing 2, and a drive head 34 having a holding arm 36 is inserted into the housing 2 through the housing side part 30. The drive head 34 grips a syringe and can be moved into and out of the housing 2 by the holding arm 36. The housing side part 30 is not mounted until the lower housing shell 32 and the upper housing shell 22 have already been assembled.

1. A medical pump, being adapted for conveying a medical fluid, the medical pump comprising:
a housing with an upper housing shell, a lower housing shell and a front lid, said front lid being pivotably arranged on the lower housing shell; and
a seal being provided between the upper housing shell and the lower housing shell, such that the housing is sealed against contamination and fluids.

2. The medical pump according to claim 1, wherein the seal is a sealing bead.

3. The medical pump according to claim 2, wherein the sealing bead is made of polyurethane.

4. The medical pump according to claim 1, wherein the seal is elastic during assembly and disassembly of the housing shells.

5. The medical pump according to claim 1, wherein the housing comprises a distance between the upper housing shell and the lower housing shell in an assembled state,
wherein the seal is placed in the distance between the upper housing shell and the lower housing shell.

6. The medical pump according to claim 1, wherein the seal is foamed.

7. The medical pump according to claim 1, wherein the seal is tempered after foaming.

8. The medical pump according to claim 1, wherein the seal extends around the entire rim of the housing shell.

9. The medical pump according to claim 1, wherein the seal is arranged inside the housing to be non-visible from the outside.

10. A peristaltic pump or a syringe pump, being adapted for conveying a medical fluid, the peristaltic pump or syringe pump comprising:
a housing with an upper housing shell, a lower housing shell and a front lid, said front lid being pivotably arranged on the lower housing shell; and
a seal being provided between the upper housing shell and the lower housing shell, such that the housing is sealed against contamination and fluids,
wherein the seal is provided on the upper housing shell as a sealing bead.

11. The peristaltic pump or syringe pump according to claim 10, wherein the sealing bead is made of polyurethane.

12. The peristaltic pump or syringe pump according to claim 10, wherein the sealing bead is elastic during assembly and disassembly of the housing shells.

13. The peristaltic pump or syringe pump according to claim 10, wherein the housing comprises a distance between the upper housing shell and the lower housing shell in an assembled state, and
wherein the sealing bead is placed in the distance between the upper housing shell and the lower housing shell.

14. The peristaltic pump or syringe pump according to claim 10, wherein the sealing bead is foamed.

15. The peristaltic pump or syringe pump according to claim 10, wherein the sealing bead is tempered after foaming.

16. The peristaltic pump or syringe pump according to claim 10, wherein the sealing bead extends around the entire rim of the housing shell.

17. The peristaltic pump or syringe pump according to claim 10, wherein the sealing bead is arranged inside the housing to be non-visible from the outside.

18. The peristaltic pump or syringe pump according to claim 10, wherein the sealing bead is provided on an upper edge of sidewalls of the upper housing shell.

19. A method of manufacturing a peristaltic pump or a syringe pump according to claim 10, comprising the following steps:
applying the seal to exactly a first housing shell of the lower housing shell and the upper housing shell;
tempering the seal; and
subsequently placing the other housing shell on the first housing shell.

20. The method according to claim 19 further comprising the step of assembling a housing side part to the composite construction of the lower housing shell and the upper housing shell.
